(19) United States
(12) Patent Application Publication
(10) Pub. No.: US 2021/0121092 A1
(43) Pub. Date: Apr. 29, 2021

(54) MAGNETIC RESONANCE IMAGING SYSTEM AND POSITION DISPLAY METHOD

(71) Applicant: CANON MEDICAL SYSTEMS CORPORATION, Otawara-shi (JP)

(72) Inventors: Sho KAWAJIRI, Nasushiobara (JP); Masashi HORI, Nasushiobara (JP)

(73) Assignee: CANON MEDICAL SYSTEMS CORPORATION, Otawara-shi (JP)

(21) Appl. No.: 17/072,713

(22) Filed: Oct. 16, 2020

(30) Foreign Application Priority Data
Oct. 29, 2019 (JP) 2019-195810

(51) Int. Cl. A61B 5/055; G01R 33/54; G01R 33/28

(52) U.S. Cl. CPC A61B 5/055; G01R 33/283; G01R 33/543

(57) ABSTRACT

A magnetic resonance imaging system according to an embodiment includes a magnetic resonance imaging device including a couch with a couchtop on which a subject is placed, and an optical photographing device. The optical photographing device acquires an image including the couch. The magnetic resonance imaging device includes a processing circuitry that performs display of information expressing a position of a first RF coil disposed under or inside the couchtop on the basis of the image acquired by the optical photographing device so that a positional relation with the subject placed on the couchtop is displayed.

### CROSS-REFERENCE TO RELATED APPLICATIONS

This application is based upon and claims the benefit of priority from Japanese Patent Application No. 2019-195810, filed on Oct. 29, 2019; the entire contents of which are incorporated herein by reference.

### FIELD

Embodiments described herein relate generally to a magnetic resonance imaging system and a position display method.

### BACKGROUND

In the conventional inspection using a magnetic resonance imaging (MRI) device, image capture is performed with RF coils for receiving magnetic resonance signals disposed near a subject. For example, in the image capture, one RF coil is disposed under or inside a couchtop on which the subject is placed, and moreover another RF coil is disposed on the subject. In such image capture, when the RF coil disposed under or inside the couchtop and the RF coil disposed on the subject are displaced from each other, an image resulting from the image capture may deteriorate in quality.

### BRIEF DESCRIPTION OF THE DRAWINGS

FIG. 1 is a diagram illustrating a structure example of an MRI system according to an embodiment;

FIG. 2 is a flowchart expressing the procedure of displaying positions of RF coils by a display controlling function according to a first example;

FIG. 3 is a diagram illustrating one example of displaying the positions of the RF coils by the display controlling function according to the first example;

FIG. 4 is a diagram illustrating another example of displaying the positions of the RF coils by the display controlling function according to the first example;

FIG. 5 is a flowchart illustrating the procedure of displaying the positions of the RF coils by the display controlling function according to a second example; and

FIG. 6 is a diagram illustrating one example of displaying the positions of the RF coils by the display controlling function according to the second example.

### DETAILED DESCRIPTION

An MRI system according to an embodiment includes an MRI device including a couch with a couchtop on which a subject is placed, and an optical photographing device. The optical photographing device acquires an image including the couch. The MRI device includes a display control unit that performs display of information expressing a position of a first RF coil disposed under or inside the couchtop on the basis of the image acquired by the optical photographing device so that a positional relation with the subject placed on the couchtop is displayed.

Embodiments of an MRI system and a position display method according to the present application are hereinafter described in detail with reference to the drawings.

### Embodiment

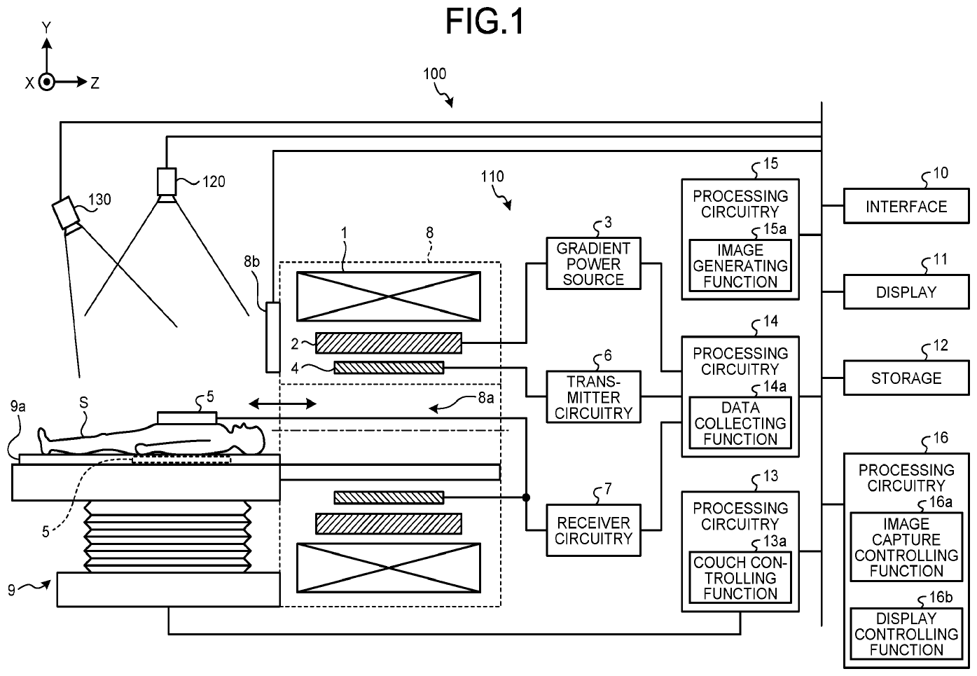

FIG. 1 is a diagram illustrating a structure example of an MRI system according to an embodiment.

For example, as illustrated in FIG. 1, an MRI system 100 includes an MRI device 110.

The MRI device 110 includes a static magnetic field magnet 1, a gradient coil 2, a gradient power source 3, a whole-body radio frequency (RF) coil 4, local RF coils 5, a transmitter circuitry 6, a receiver circuitry 7, a gantry 8, a couch 9, an interface 10, a display 11, a storage 12, and processing circuitries 13 to 16.

The static magnetic field magnet 1 generates a static magnetic field in an image capture space where a subject S is placed. Specifically, the static magnetic field magnet 1 is formed to have a hollow, approximately cylindrical shape (including the shape whose cross section orthogonal to a central axis is elliptical) and generates a static magnetic field in the image capture space on an inner peripheral side thereof. For example, the static magnetic field magnet 1 is a superconducting magnet, a permanent magnet, or the like. The superconducting magnet herein referred to includes, for example, a vessel filled with a cooling agent such as liquid helium, and a superconducting coil immersed in the vessel.

The gradient coil 2 is disposed inside the static magnetic field magnet 1, and generates a gradient field in the image capture space where the subject S is placed. Specifically, the gradient coil 2 is formed to have a hollow, approximately cylindrical shape (including the shape whose cross section orthogonal to the central axis is elliptical) and includes an X coil, a Y coil, and a Z coil that correspond to an X axis, a Y axis, and a Z axis that are orthogonal to each other. The X coil, the Y coil, and the Z coil generate, in the image capture space, the gradient fields that change linearly along the respective axial directions on the basis of the current supplied from the gradient power source 3. Here, the Z axis is set to extend along the magnetic flux of the static magnetic field that is generated by the static magnetic field magnet 1. In addition, the X axis is set to extend along a horizontal direction that is orthogonal to the Z axis, and the Y axis is set to extend along a vertical direction that is orthogonal to the Z axis. Thus, the X axis, the Y axis, and the Z axis form a device coordinate system that is unique to the MRI device 110.

The gradient power source 3 generates the gradient field in the image capture space by supplying current to the gradient coil 2. Specifically, the gradient power source 3 supplies current to each of the X coil, the Y coil, and the Z coil of the gradient coil 2, so that the gradient field that changes linearly along a readout direction, a phase encode direction, and a slice direction, which are orthogonal to each other, is generated in the image capture space. Note that the gradient field along the readout direction is referred to as a readout gradient field, the gradient field along the phase encode direction is referred to as a phase encode gradient field, and the gradient field along the slice direction is referred to as a slice gradient field.

Here, when each of the readout gradient field, the phase encode gradient field, and the slice gradient field is overlapped with the static magnetic field generated by the static magnetic field magnet 1, the spatial positional information is added to a magnetic resonance signal that is generated from the subject S. Specifically, the readout gradient field adds the positional information along the readout direction to the magnetic resonance signal by changing the frequency of the magnetic resonance signal in accordance with the position in the readout direction. In addition, the phase encode gradient field adds the positional information along the phase encode direction to the magnetic resonance signal by changing the phase of the magnetic resonance signal along the phase encode direction. Furthermore, the slice gradient field adds the positional information along the slice direction to the magnetic resonance signal. For example, when an image capture region is a slice region (2D image), the slice gradient field is used to determine the direction, the thickness, and the number of slice regions, and when the image capture region is a volume region (3D image), the slice gradient field is used to change the phase of the magnetic resonance signal in accordance with the position in the slice direction. Thus, the axis along the readout direction, the axis along the phase encode direction, and the axis along the slice direction form a logical coordinate system that defines the slice region or the volume region to be an object of the image capture.

The whole-body RF coil 4 is disposed on an inner peripheral side of the gradient coil 2, transmits a high-frequency magnetic field to the subject S disposed in the image capture space, and receives the magnetic resonance signal generated from the subject S due to the influence from the high-frequency magnetic field. Specifically, the whole-body RF coil 4 is formed to have a hollow, approximately cylindrical shape (including the shape whose cross section orthogonal to the central axis is elliptical), and transmits the high-frequency magnetic field to the subject S disposed in the image capture space positioned on the inner peripheral side thereof on the basis of a high-frequency pulse signal supplied from the transmitter circuitry 6. In addition, the whole-body RF coil 4 receives the magnetic resonance signal generated from the subject S due to the influence from the high-frequency magnetic field, and outputs the received magnetic resonance signal to the receiver circuitry 7.

The local RF coils 5 receive the magnetic resonance signals generated from the subject S. Specifically, a plurality of kinds of local RF coils 5 are prepared so as to be placed at each part of the subject S, and are each disposed near a surface of the part to be the object of the image capture. The local RF coils 5 receive the magnetic resonance signals generated from the subject S due to the influence from the high-frequency magnetic field transmitted from the whole-body RF coil 4, and output the received magnetic resonance signals to the receiver circuitry 7. Note that the local RF coil 5 may further have a function of transmitting the high-frequency magnetic field to the subject S. In that case, the local RF coil 5 is connected to the transmitter circuitry 6, and transmits the high-frequency magnetic field to the subject S on the basis of the high-frequency pulse signal supplied from the transmitter circuitry 6. For example, the local RF coil 5 is a surface coil or a phased array coil combining a plurality of surface coils as elements.

The transmitter circuitry 6 outputs to the whole-body RF coil 4, a high-frequency pulse signal corresponding to the Larmor frequency unique to the object atomic nucleus placed in the static magnetic field. Specifically, the transmitter circuitry 6 includes a pulse generator, a high-frequency generator, a modulator, and an amplifier. The pulse generator generates the waveform of the high-frequency pulse signal. The high-frequency generator generates the high-frequency signal with the Larmor frequency. The modulator generates the high-frequency pulse signal by modulating the amplitude of the high-frequency signal generated from the high-frequency generator with the waveform generated from the pulse generator. The amplifier amplifies the high-frequency pulse signal generated by the modulator, and outputs the amplified signal to the whole-body RF coil 4.

The receiver circuitry 7 generates the magnetic resonance data on the basis of the magnetic resonance signals output from the whole-body RF coil 4 or the local RF coils 5, and outputs the generated magnetic resonance data to the processing circuitry 14. For example, the receiver circuitry 7 includes a selector, a preamplifier, a phase detector, and an A/D (analog/digital) converter. The selector selectively inputs the magnetic resonance signal output from the whole-body RF coil 4 or the local RF coil 5. The preamplifier amplifies the power of the magnetic resonance signal output from the selector. The phase detector detects the phase of the magnetic resonance signal output from the preamplifier. The A/D converter generates the magnetic resonance data by converting an analog signal output from the phase detector to a digital signal, and outputs the generated magnetic resonance data to the processing circuitry 14. Note that each process, which is performed by the receiver circuitry 7 in the above description, is not necessarily performed by the receiver circuitry 7 entirely and a part of the process (for example, the process by the A/D converter) may be performed in the whole-body RF coil 4 or the local RF coil 5.

The gantry 8 includes a hollow bore 8a which is formed by an approximately cylindrical shape (including the shape whose cross section orthogonal to the central axis is elliptical), and houses the static magnetic field magnet 1, the gradient coil 2, and the whole-body RF coil 4. Specifically, the gantry 8 houses the whole-body RF coil 4, the gradient coil 2, and the static magnetic field magnet 1 while the whole-body RF coil 4 is disposed on the outer peripheral side of the bore 8a, the gradient coil 2 is disposed on the outer peripheral side of the whole-body RF coil 4, and the static magnetic field magnet 1 is disposed on the outer peripheral side of the gradient coil 2. Here, the space in the bore 8a of the gantry 8 is the image capture space where the subject S is disposed at the image capture.

The couch 9 includes a couchtop 9a where the subject S is placed, and a movement mechanism that moves the couchtop 9a in an up-down direction and the horizontal direction. Here, the up-down direction is a vertical direction, and the horizontal direction is a direction along the central axis of the static magnetic field magnet 1. Such a structure can change the height of the couchtop 9a of the couch 9 by moving the couchtop 9a in the up-down direction. In addition, as the couchtop 9a is moved in the horizontal direction, the position of the couchtop 9a of the couch 9 can be changed between the space outside the gantry 8 and the image capture space in the bore 8a inside the gantry 8.

The static magnetic field magnet 1, the gradient coil 2, and the whole-body RF coil 4 in the MRI device 110 are formed to have the approximately cylindrical shape, which is a so-called tunnel type structure, is described here; however, the embodiment is not limited to this structure. For example, the MRI device 110 may have a pair of static magnetic field magnets, a pair of gradient coils, and a pair of RF coils so as to have the image capture space, where the subject S is placed, therebetween, which is a so-called open type structure. In such an open type structure, the space between the pair of static magnetic field magnets, the pair of gradient coils, and the pair of RF coils corresponds to the bore in the tunnel type structure.

The interface 10 receives various instructions or the input operation of various pieces of information from the operator. Specifically, the interface 10 is connected to the processing circuitry 16, and converts the input operation received from the operator into an electric signal and outputs the resulting signal to the processing circuitry 16. For example, the interface 10 is formed by a trackball, a switch button, a mouse, or a keyboard for setting an image capture condition or a region of interest (ROI), a touchpad where the input operation is performed by the touch on the operation surface, a touchscreen combining a display screen and a touch pad, a noncontact input circuitry including an optical sensor, a sound input circuitry, or the like. Note that the interface 10 herein described does not just mean the physical operation component such as a mouse or a keyboard. Another example of the interface 10 is a processing circuitry that receives, from an external input device provided separately from the device, an electric signal corresponding to the input operation and outputs this electric signal to a control circuitry.

The display 11 displays various kinds of information and various images. Specifically, the display 11 is connected to the processing circuitry 16, and converts various kinds of information and the data of various kinds of images that are transmitted from the processing circuitry 16 into an electric signal for display, and outputs the resulting electric signal. For example, the display 11 is achieved by a liquid crystal monitor, a cathode ray tube (CRT) monitor, a touch panel, or the like.

The storage 12 stores various kinds of data. Specifically, the storage 12 stores the magnetic resonance data and the image data. For example, the storage 12 is achieved by a semiconductor memory element such as a random access memory (RAM) or a flash memory, a hard disk, an optical disk, or the like.

The processing circuitry 13 includes a couch controlling function 13a. The couch controlling function 13a controls the operation of the couch 9 by outputting a control electric signal to the couch 9. For example, the couch controlling function 13a receives the operator's instruction for moving the couchtop 9a in the up-down direction or the horizontal direction through the interface 10 or an operation panel provided to the gantry 8, and operates the movement mechanism in the couch 9 so as to move the couchtop 9a in accordance with the received instruction. For example, the couch controlling function 13a moves the couchtop 9a where the subject S is placed to the image capture space in the bore 8a inside the gantry 8 when the image of the subject S is captured.

The processing circuitry 14 has a data collecting function 14a. The data collecting function 14a collects the magnetic resonance data of the subject S by executing various pulse sequences. Specifically, the data collecting function 14a performs various pulse sequences by driving the gradient power source 3, the transmitter circuitry 6, and the receiver circuitry 7 in accordance with the sequence executing data output from the processing circuitry 16. Here, the sequence executing data is the data expressing the pulse sequence, and is the information that defines the timing when the gradient power source 3 supplies the current to the gradient coil 2, the amount of supplied current, the timing when the transmitter circuitry 6 supplies the high-frequency pulse signal to the whole-body RF coil 4, the intensity of the supplied high-frequency pulse, the timing when the receiver circuitry 7 samples the magnetic resonance signal, or the like. Then, the data collecting function 14a receives the magnetic resonance data output from the receiver circuitry 7 as a result of executing the pulse sequence, and causes the storage 12 to store the magnetic resonance data. Here, the magnetic resonance data to be stored in the storage 12 is stored as the data expressing the two-dimensional or three-dimensional k space in which the positional information along each of the readout direction, the phase encode direction, and the slice direction depending on the readout gradient field, the phase encode gradient field, and the slice gradient field that are described above is added.

The processing circuitry 15 has an image generating function 15a. The image generating function 15a generates various images on the basis of the magnetic resonance data collected by the processing circuitry 14. Specifically, the image generating function 15a reads out the magnetic resonance data collected by the processing circuitry 14 from the storage 12, and performs a reconfiguring process, such as Fourier transformation, on the read magnetic resonance data, so as to generate the two-dimensional or three-dimensional image. Then, the image generating function 15a causes the storage 12 to store the generated image.

The processing circuitry 16 has an image capture controlling function 16a. The image capture controlling function 16a controls each component in the MRI device 110 so as to control the entire MRI device 110. Specifically, the image capture controlling function 16a causes the display 11 to display the graphical user interface (GUI) to receive various instructions and the input operation of various kinds of information from the operator, and controls each component in the MRI device 110 in accordance with the input operation received through the interface 10. For example, the image capture controlling function 16a generates the sequence executing data on the basis of the image capture condition input by the operator, and outputs the generated sequence executing data to the processing circuitry 14, thereby collecting the magnetic resonance data. In another example, the image capture controlling function 16a controls the processing circuitry 15 so as to generate the image on the basis of the magnetic resonance data collected by the processing circuitry 14. In still another example, the image capture controlling function 16a reads out the image stored in the storage 12 and causes the display 11 to display the read image in accordance with the request from the operator.

Here, each processing circuitry is achieved by the processor, for example. In this case, the processing function of each processing circuitry is stored in the storage 12 in the form of a computer-executable computer program. Each processing circuitry reads out and executes the computer program from the storage 12 so as to achieve the processing function corresponding to each computer program. In other words, each processing circuitry that has read out the computer program has each function indicated in each processing circuitry in FIG. 1.

Each processor is achieved by a single processor in the above description; however, each processing circuitry may be formed by combining a plurality of independent processors and each processing function may be achieved by causing each processor to execute the computer program. Alternatively, the processing function of each processing circuitry may be achieved by diffusing or integrating the functions as appropriate in one or more processing circuitries. In the example illustrated in FIG. 1, one storage 12 stores the computer program corresponding to each processing function; however, storages may be diffusedly disposed and the processing circuitry may be configured to read out the corresponding computer program from the individual storage.

The components of the MRI device 110 described above are disposed separately in a photographing room configured as a shield room that shields the internal space from electromagnetic waves, and an operation room where the operation for the MRI device 110 is performed. For example, the static magnetic field magnet 1, the gradient coil 2, the whole-body RF coil 4, the local RF coils 5, the receiver circuitry 7, the gantry 8, the couch 9, and the processing circuitry 13 are disposed in the photographing room, and the gradient power source 3, the transmitter circuitry 6, the interface 10, the display 11, the storage 12, and the processing circuitries 14 to 16 are disposed in the operation room. In a case where a machine room is further provided in addition to the photographing room and the operation room, the gradient power source 3, the transmitter circuitry 6, the storage 12, and the processing circuitries 14 to 16 may be partially or entirely disposed in the machine room.

The entire structure of the MRI device 110 according to the present embodiment has been described. In this structure, when the subject S is inspected, the MRI device 110 according to the present embodiment captures the image of the subject S so as to collect the images necessary for the inspection.

Here, in the usual inspection using the MRI device, the RF coil to receive the magnetic resonance signal is disposed near the subject to capture the image. For example, the RF coil is disposed under or inside the couchtop on which the subject is placed, and moreover the RF coil is disposed on the subject before the image capture. In this image capture, if the RF coil disposed under or inside the couchtop and the RF coil disposed on the subject are displaced from each other, the image resulting from the image capture may deteriorate in quality.

For example, in parallel imaging, which is one of the fast imaging methods performed by the MRI device, a spine coil (RF coil for imaging the spine) is disposed under or inside the couchtop, and moreover a body coil (RF coil for imaging the abdomen) is disposed on the subject; thus, the image capture is performed. Here, usually, the spine coil and the body coil are each formed by a plurality of elements and if the elements are displaced from each other when the coils are disposed, the image quality of the parallel imaging may deteriorate.

In view of the above, the MRI system 100 according to the present embodiment is configured so that the RF coils can be set at the proper positions.

Specifically, the MRI system 100 includes a camera 120 disposed on the couch 9. For example, the camera 120 is attached to the ceiling of the photographing room. In addition, the camera 120 may be attached to an end of the gantry 8 or the couch 9, or to the wall near the gantry 8 or the couch 9. Note that the camera 120 is one example of the optical photographing device.

The MRI system 100 includes a projector 130 disposed above the couch 9. For example, the projector 130 is attached to the ceiling of the photographing room. The projector 130 may be attached to the end of the gantry 8, or to the wall near the gantry 8 or the couch 9.

In addition, the gantry 8 of the MRI device 110 included in the MRI system 100 includes a gantry monitor 8b, and the processing circuitry 16 has a display controlling function 16b. Note that the display controlling function 16b is one example of the display control unit.

In the present embodiment, the camera 120 acquires an image including the couch 9, and the display controlling function 16b performs the display of information expressing the position of the local RF coil 5 (first RF coil) disposed under or inside the couchtop 9a on the basis of the image acquired by the camera 120 so that the positional relation with the subject S on the couchtop 9a is displayed.

In the present embodiment, the display controlling function 16b further performs the display of the information expressing the recommended position to dispose the local RF coil 5 (second coil), which is disposed on a patient, so that the positional relation with the patient placed on the couchtop 9a is displayed.

In such a structure, when a technologist sets the local RF coil 5 on the subject, the technologist can find the position of the local RF coil 5 that is disposed under or inside the couchtop 9a even if the subject exists on the couchtop 9a. Accordingly, the RF coil can be disposed at the proper position in the present embodiment.

Specific application examples of the MRI system 100 according to the present embodiment are described as examples. In the examples to be described below, the subject S is a patient. In the examples below, the technologist's work of attaching or detaching the coil to or from the couchtop 9a and the patient is called “coil setting”.

Moreover, in the examples below, the local RF coil 5 (first RF coil) to be disposed under or inside the couchtop 9a is the spine coil (RF coil for imaging the spine) and the local RF coil 5 (second RF coil) to be disposed on the patient is the body coil (RF coil for imaging the abdomen). Here, each of the spine coil and the body coil includes a plurality of elements.

### First Example

Here, a first example is described. In the first example, the display controlling function 16b causes the gantry monitor 8b to display the information expressing the position of the spine coil, which is disposed under or inside the couchtop 9a, while overlapping this information with the image of the patient acquired by the camera 120.

Moreover, in the first example, the display controlling function 16b causes the gantry monitor 8b to display the information expressing the recommended position to dispose the body coil, which is disposed on the patient, while further overlapping this information with the image of the patient acquired by the camera 120.

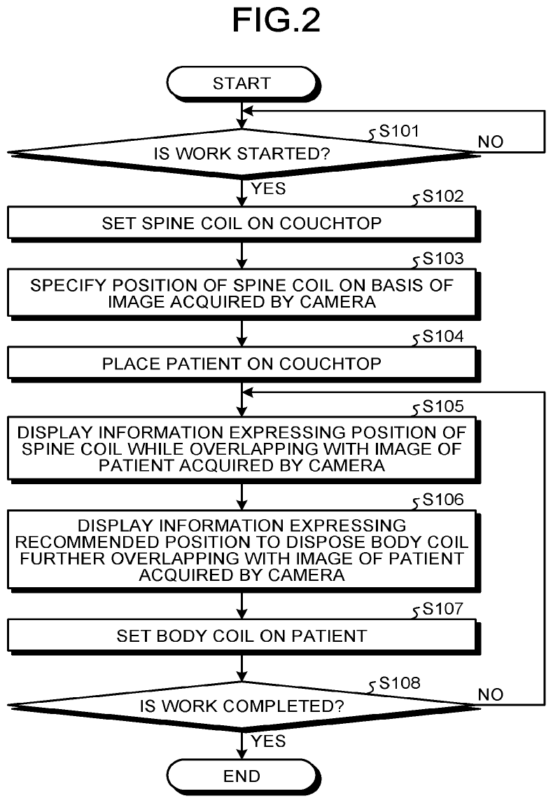

FIG. 2 is a flowchart expressing the procedure of displaying the positions of the RF coils by the display controlling function 16b according to the first example.

For example, as illustrated in FIG. 2, upon the start of the coil setting work (step S101), first, the technologist sets the spine coil on the couchtop 9a (step S102).

After that, the display controlling function 16b specifies the position of the spine coil on the basis of the image acquired by the camera 120 (step S103).

The present example is very advantageous in that the work of optimizing the positional relation between the elements of the spine coil on the back of the patient and the body coil on the abdomen of the patient can be reduced. In the description below, a case in which the patient lies down on the couchtop 9a of the couch 9 on his back is assumed.

In particular, when the patient exists on the couchtop 9a, it is difficult to specify the position of the element of the spine coil. In the actual workflow, the technologist does not want to make the patient get up from the couch and find the position of the spine coil. Therefore, it is important for the system to grasp the position of the element of the spine coil when the patient does not exist on the couchtop 9a.

Note that in a case where the spine coil is embedded in the couchtop 9a, the position where the spine coil is disposed in the couchtop 9a is uniquely determined; therefore, this procedure (step S103) is unnecessary. On the other hand, in a case where the spine coil can be moved to an arbitrary position, it is necessary to grasp the position of the spine coil in the couch in advance.

In view of this, the display controlling function 16b specifies the position of the couchtop 9a and the spine coil on the basis of the image acquired by the camera 120 after the technologist sets the spine coil under or inside the couchtop 9a and before the patient gets on the couchtop 9a.

For example, the display controlling function 16b specifies the positions of the couchtop 9a and the spine coil using one of the following three methods.

Method 1) The image is captured using the camera 120 at the timing when lowering of the couchtop 9a is detected, and using the obtained image, the positions of the couchtop 9a and the spine coil are specified. This is because, after the spine coil is set, the couchtop 9a is lowered so that the patient gets thereon.

Method 2) After the spine coil is set, the image capture is performed continuously by the camera 120 and at the timing when the technologist goes out of a frame of the image capture region of the camera 120, the positions of the couchtop 9a and the spine coil are specified using the previously obtained image. This is because, after the spine coil is set, the technologist goes out of the photographing room once to guide the patient to the photographing room.

Method 3) After the spine coil is set, the image capture is performed continuously at a low frame rate (for example, every one second, every five seconds, etc.) by the camera 120, and at the timing when the patient comes in a frame of the image of the couchtop 9a, the positions of the couchtop 9a and the spine coil are specified using the image obtained just before the patient comes in the frame of the image.

Subsequently, the patient is placed on the couchtop 9a by the technologist (step S104).

After that, the display controlling function 16b causes the gantry monitor 8b to display the information expressing the position of the spine coil while overlapping this information with the image of the patient acquired by the camera 120 (step S105). The display controlling function 16b causes the gantry monitor 8b to display the information expressing the recommended position to dispose the body coil while overlapping this information with the image of the patient acquired by the camera 120 (step S106).

Here, the display controlling function 16b performs the display of the information expressing the positions of all the elements included in the spine coil within the information expressing the position of the spine coil. Moreover, the display controlling function 16b performs the display of the information expressing the positions of all the elements included in the body coil within the information expressing the recommended position to dispose the body coil.

Subsequently, the body coil is set on the patient by the technologist (step S107).

Here, the display controlling function 16b performs the display of the information expressing the position of the spine coil and the information expressing the recommended position to dispose the body coil so that the positional relation with the body coil that is actually disposed on the patient is displayed.

Specifically, the display controlling function 16b causes the gantry monitor 8b to keep displaying the image of the patient who is photographed by the camera 120, so that the information expressing the position of the spine coil and the information expressing the recommended position of the body coil are displayed in a manner of overlapping with the image of the body coil that is actually disposed on the patient. Here, the frame rate of the display and the image acquisition from the camera 120 is not specified; however, it is desirable that the display is switched at the speed of such a degree that the image looks like a moving image when the technologist sees the gantry monitor 8b.

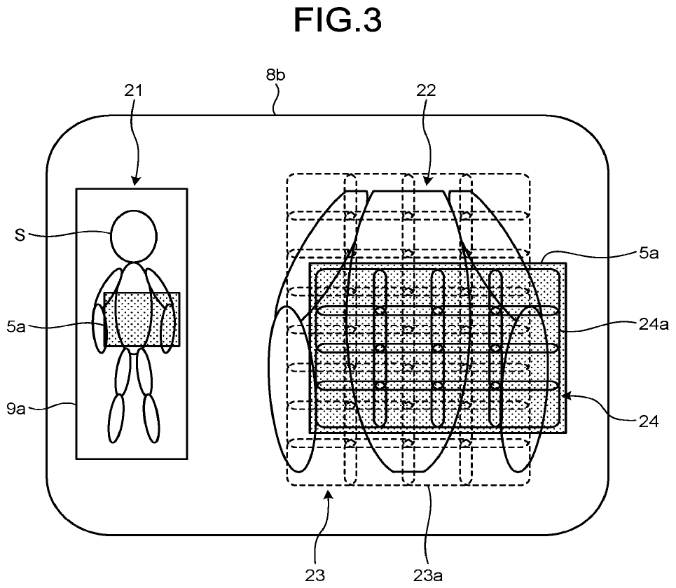

FIG. 3 is a diagram illustrating one example of displaying the positions of the RF coils by the display controlling function 16b according to the first example.

For example, as illustrated in FIG. 3, the display controlling function 16b causes the gantry monitor 8b to display a first image 21 and a second image 22 that are acquired by the camera 120. The first image 21 includes the whole body of the patient S on the couchtop 9a and the body coil 5a actually set on the patient S. The second image 22 is a magnified image of the range where the spine coil is disposed in the first image 21.

Then, the display controlling function 16b performs the display of a mark 23 expressing the position of the spine coil and a mark 24 expressing the recommended position of the body coil in a manner overlapping with the second image 22. For example, the display controlling function 16b performs the display of a mark expressing a corner or a center of the coil, a frame-shaped mark expressing an edge of the coil, or the like, as the mark 23 expressing the position of the spine coil and the mark 24 expressing the recommended position of the body coil.

Here, for example, the display controlling function 16b determines the recommended position of the body coil using the following method.

Method 1) The recommended position is determined so that the center of the coil comes to a particular part of the patient on the basis of the information about the anatomy (captured part) selection included in the image capture condition.

Method 2) The recommended position is determined so that the positional relation between the spine coil and the body coil is optimized on the basis of the information about the element specified in the image capture condition.

Method 3) The recommended position is determined so that the positional relation between the element of the spine coil and the element of the body coil is optimized. For example, the recommended position is determined so that the center of the element of the spine coil and the center of the body coil coincide.

Any of the aforementioned methods may be used alone or a plurality of the methods may be used in combination. Alternatively, a method selected by the operator may be used.

Furthermore, the display controlling function 16b performs the display of a mark 23a expressing the position of each element included in the spine coil within the mark 23 expressing the position of the spine coil. Moreover, the display controlling function 16b performs the display of a mark 24a expressing the position of each element included in the body coil within the mark 24 expressing the recommended position of the body coil. For example, the display controlling function 16b performs the display of a mark expressing a corner or a center of the element, a frame-shaped mark expressing an edge of the element, or the like, for each element as the mark 23a expressing the position of each element included in the spine coil and the mark 24a expressing the position of each element included in the body coil.

Thus, the technologist can dispose the body coil at the optimal position by working while checking the information expressing the position of the spine coil, the information expressing the recommended position of the body coil, and the image of the body coil that is actually disposed, which are displayed on the gantry monitor 8b.

Here, for example, in a case where the experienced technologist sets the coil, he usually finds the position of the element and then sets the coil so that the element comes to the optimal position for the body part that he wants to photograph. In this case, as described above, it is effective to display the information expressing the position of the element of each of the spine coil and the body coil.

Even when the patient lies down, the position of the element of the spine coil can be found with the gantry monitor 8b; therefore, the position of the patient can be minutely adjusted with respect to the position of the element of the spine coil.

The spine coil and the body coil can be disposed so that their elements are overlapped with each other. Thus, the elements can be disposed at the positions where the developing performance of the parallel imaging is improved and therefore, the RF coils can be positioned so as to maximize the performance of the parallel imaging. For example, in the case where the parallel imaging is performed, the display controlling function 16b may calculate a g-factor (index expressing the developing performance of the parallel imaging) from the positional relation between the spine coil and the body coil, and further cause the gantry monitor 8b to display the information expressing the calculated g-factor.

Thus, while the coil setting is performed (step S108), the display of the position of the spine coil and the display of the recommended position of the body coil by the display controlling function 16b and the setting of the body coil by the technologist are repeatedly performed (steps S105 to S107).

Here, in the aforementioned procedure, the process in steps S103, S105, and S106 is performed when, for example, the processing circuitry 16 reads out a predetermined computer program corresponding to the display controlling function 16b from the storage 12 and executes the computer program.

Note that in the first example described above, the display controlling function 16b performs the display of the information expressing the positions of all the elements included in the spine coil and the body coil; however, the example is not limited to this structure.

For example, the display controlling function 16b may perform the display of the information expressing the position of a part of the elements included in the spine coil. Alternatively, the display controlling function 16b may perform the display of the information expressing the position of a part of the elements included in the body coil.

For example, in a case where the MRI device 110 has a function of selecting the element used in the image capture in the unit called a segment in which some elements included in the RF coil are grouped, the display controlling function 16b may perform the display of the information expressing the position of the selected segment. For example, in a case where the elements are disposed in matrix in the spine coil and the body coil, the elements included in the same row or the elements included in the same column are grouped as one segment.

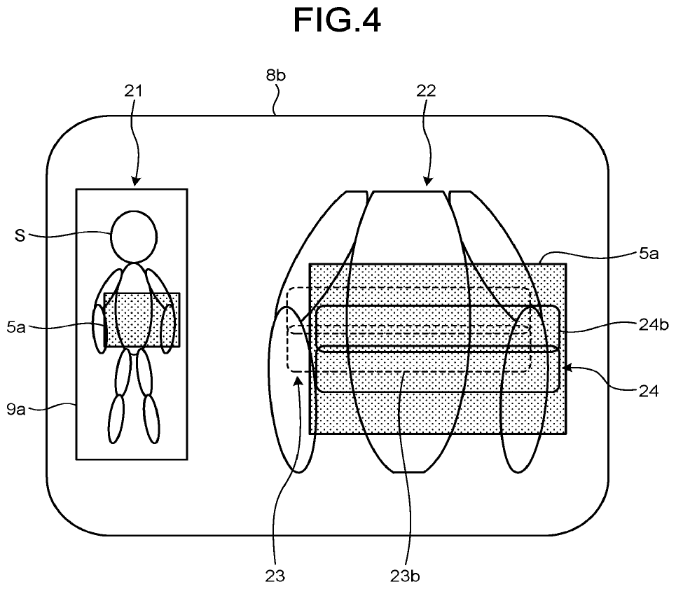

FIG. 4 is a diagram illustrating another example of displaying the positions of the RF coils by the display controlling function 16b according to the first example.

For example, in a manner similar to the example illustrated in FIG. 3, the display controlling function 16b causes the gantry monitor 8b to display the first image 21 and the second image 22 that are acquired by the camera 120 as illustrated in FIG. 4. The first image 21 includes the whole body of the patient S placed on the couchtop 9a and the body coil 5a actually disposed on the patient S, and the second image 22 is the image magnifying the range where the spine coil is disposed in the first image 21. The display controlling function 16b performs the display of a mark 23 expressing the position of the spine coil and a mark 24 expressing the recommended position of the body coil in a manner of overlapping with the second image 22, which is similar to the example illustrated in FIG. 3.

Here, the display controlling function 16b performs the display of a mark 23b expressing the position of the segment selected in the spine coil within the mark 23 expressing the position of the spine coil overlapped with the second image 22. In addition, the display controlling function 16b performs the display of a mark 24b expressing the position of the segment selected in the body coil within the mark 24 expressing the recommended position of the body coil overlapped with the second image 22. For example, the display controlling function 16b performs the display of a mark expressing a corner or a center of the segment, a frame-shaped mark expressing an edge of the segment, or the like, for each segment as the mark 23b expressing the position of the segment selected in the spine coil and the mark 24b expressing the position of the segment selected in the body coil.

Alternatively, in a case where the MRI device 110 has a function of selecting the element used in the image capture in the unit of element, the display controlling function 16b may perform the display of the information expressing the position of the selected element among the elements included in the spine coil and the body coil.

Alternatively, for example, the display controlling function 16b may perform the display of only the information about the position of a part of the elements included in the spine coil and the body coil that is near the edge of the coils.

For example, some manufacturers of the MRI device 110 may be concerned about displaying the information about all the elements because, for example, the structure of the element in the RF coil contains their original technology. In this case, as described above, it is effective to display the information expressing the position of a part of the elements included in the spine coil or the body coil.

In the first example described above, the display controlling function 16b causes the gantry monitor 8b to display the image acquired by the camera 120, the information expressing the position of the spine coil, and the information expressing the recommended position of the body coil; however, the example is not limited to this structure.

For example, the display controlling function 16b may cause a monitor provided at the couch 9, a portable monitor, a monitor of a mobile terminal used by the technologist, or the like, to display the image acquired by the camera 120, the information expressing the position of the spine coil, and the information expressing the recommended position of the body coil.

### Second Example

Next, a second example is described. In the second example, the display controlling function 16b causes the projector 130 to perform display of the information expressing the position of the spine coil disposed under or inside the couchtop 9a by projecting the information on the patient placed on the couchtop 9a.

In the second example, regarding the body coil to be disposed on the patient, the display controlling function 16b further causes the projector 130 to project the information expressing the recommended position to dispose the body coil onto the patient placed on the couchtop 9a, and display the information.

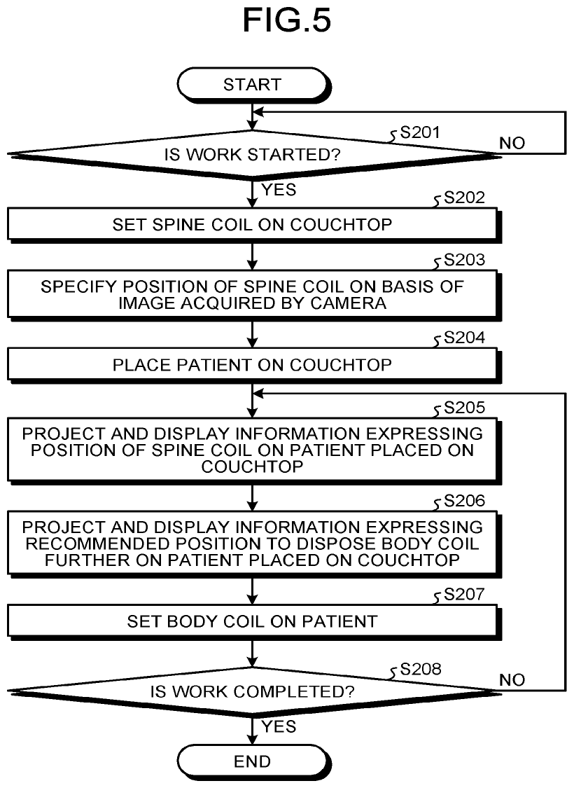

FIG. 5 is a flowchart illustrating the procedure of displaying the positions of the RF coils by the display controlling function 16b according to the second example.

For example, as illustrated in FIG. 5, upon the start of the coil setting work (step S201), first, the technologist sets the spine coil on the couchtop 9a (step S202).

After that, the display controlling function 16b specifies the position of the spine coil on the basis of the image acquired by the camera 120 (step S203). For example, the display controlling function 16b specifies the position of the spine coil by a method similar to that in the first example.

Subsequently, the patient is placed on the couchtop 9a by the technologist (step S204).

After that, the display controlling function 16b causes the projector 130 to perform display of the information expressing the position of the spine coil by projecting the information onto the patient placed on the couchtop 9a (step S205). In addition, the display controlling function 16b further causes the projector 130 to project the information expressing the recommended position to dispose the body coil onto the patient placed on the couchtop 9a, and display the information (step S206).

Here, the display controlling function 16b performs the display of the information expressing the positions of all the elements included in the spine coil within the information expressing the position of the spine coil in a manner similar to the case in the first example. Moreover, the display controlling function 16b performs the display of the information expressing the positions of all the elements included in the body coil within the information expressing the recommended position to dispose the body coil in a manner similar to the case in the first example.

Subsequently, the body coil is set on the patient by the technologist (step S207).

Here, the display controlling function 16b performs the display of the information expressing the position of the spine coil and the information expressing the recommended position to dispose the body coil so that the positional relation with the body coil that is actually disposed on the patient is displayed.

Specifically, the display controlling function 16b keeps projecting, onto the patient, the information expressing the position of the spine coil and the information expressing the recommended position of the body coil, so that the information expressing the position of the spine coil and the information expressing the recommended position of the body coil are displayed on the body coil that is actually disposed on the patient.

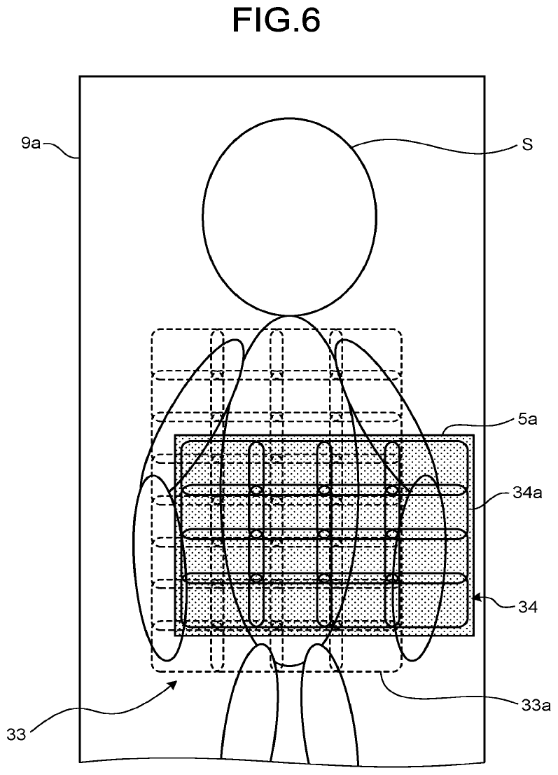

FIG. 6 is a diagram illustrating one example of displaying the positions of the RF coils by the display controlling function 16b according to the second example.

For example, as illustrated in FIG. 6, the display controlling function 16b performs the display of a mark 33 expressing the position of the spine coil and a mark 34 expressing the recommended position of the body coil through the projection on the patient S placed on the couchtop 9a and the body coil 5a that is actually disposed on the patient S. For example, the display controlling function 16b performs the display of a mark expressing a corner or a center of the coil, a frame-shaped mark expressing an edge of the coil, or the like, as the mark 33 expressing the position of the spine coil and the mark 34 expressing the recommended position of the body coil.

Here, for example, the display controlling function 16b determines the recommended position of the body coil in a manner similar to the method in the first example.

Furthermore, the display controlling function 16b performs the display of a mark 33a expressing the position of each element included in the spine coil within the mark 33 expressing the position of the spine coil. In addition, the display controlling function 16b performs the display of a mark 34a expressing the position of each element included in the body coil within the mark 34 expressing the recommended position of the body coil. For example, the display controlling function 16b performs the display of a mark expressing a corner or a center of the element, a frame-shaped mark expressing an edge of the element, or the like, for each element as the mark 33a expressing the position of each element included in the spine coil and the mark 34a expressing the position of each element included in the body coil.

Thus, the technologist can dispose the body coil at the optimal position by working while checking the information expressing the position of the spine coil displayed on the patient, the information expressing the recommended position of the body coil, and the position of the body coil that is actually disposed.

Here, for example, in a case where an experienced technologist sets the coil, he usually finds the position of the element and then sets the coil so that the element comes to the optimal position for the body part that he wants to photograph. In this case, as described above, it is effective to display the information expressing the position of the element of each of the spine coil and the body coil.

Even when the patient lies down, the position of the element of the spine coil can be found with the gantry monitor 8b; therefore, the position of the patient can be minutely adjusted with respect to the position of the element of the spine coil.

The spine coil and the body coil can be disposed so that their elements are overlapped with each other. Thus, the elements can be disposed at the positions where the developing performance of the parallel imaging is improved and therefore, the RF coils can be positioned so as to maximize the performance of the parallel imaging. For example, in the case where the parallel imaging is performed, the display controlling function 16b may calculate the g-factor from the positional relation between the spine coil and the body coil, and further project the information expressing the calculated g-factor onto the patient and display the information.

Thus, while the coil setting is performed (step S208), the display of the position of the spine coil and the display of the recommended position of the body coil by the display controlling function 16b and the setting of the body coil by the technologist are repeatedly performed (steps S205 to S207).

Here, in the aforementioned procedure, the process in steps S203, S205, and S206 is performed when, for example, the processing circuitry 16 reads out a predetermined computer program corresponding to the display controlling function 16b from the storage 12 and executes the computer program.

Note that in the second example described above, the display controlling function 16b performs the display of the information expressing the positions of all the elements included in the spine coil and the body coil; however, the example is not limited to this structure.

For example, the display controlling function 16b may perform the display of the information expressing the position of a part of the elements included in the spine coil. Alternatively, the display controlling function 16b may perform the display of the information expressing the position of a part of the elements included in the body coil.

For example, the display controlling function 16b may perform the display of the information expressing the position of the selected segment, the information expressing the position of the selected element, the information expressing the position of the element near the edge of the coil, or the like, by a method similar to that in the first example.

In the examples described above, the display controlling function 16b performs the display of the information expressing the position of the spine coil and the information expressing the recommended position of the body coil; however, the example is not limited to this structure.

For example, the display controlling function 16b may calculate the displacement between the body coil actually disposed on the patient and the recommended position of the body coil on the basis of the image acquired by the camera 120, and further offer the information expressing the calculated amount of displacement. In this case, the display controlling function 16b may cause the projector 130 to project the information expressing the calculated amount of displacement onto the patient, the gantry 8, or the couch 9, or output the information with a sound.

In the aforementioned examples, the local RF coil 5 to be disposed on the patient is the body coil; however, the examples are not limited to this structure. In another example, the local RF coil 5 that is disposed on the patient may be the RF coil for another part, such as an RF coil for a leg part or an RF coil for a head part.

The examples of the MRI system 100 according to the present embodiment have been described. As described in the above examples, in the present embodiment, the camera 120 acquires the image including the couch 9, and the display controlling function 16b performs the display of the information expressing the position of the local RF coil 5 disposed under or inside the couchtop 9a on the basis of the image acquired by the camera 120 so that the positional relation with the subject S placed on the couchtop 9a is displayed.

In this structure, when the technologist works to set the local RF coil 5 on the subject, the technologist can find the position of the local RF coil 5 that is disposed under or inside the couchtop 9a even if the subject exists on the couchtop 9a. Accordingly, the RF coil can be disposed at the proper position in the present embodiment.

In the present embodiment, regarding the local RF coil 5 (second coil) to be disposed on the patient, the display controlling function 16b further performs the display of the information expressing the recommended position to dispose the local RF coil 5 so that the positional relation with the subject placed on the couchtop 9a is displayed.

In this structure, the technologist can be navigated so that the position of the element of the local RF coil 5 to be disposed on the patient is optimized for the position of the local RF coil 5 that is disposed under or inside the couchtop 9a.

Furthermore, before the image capture, the position of the local RF coil 5 can be adjusted as appropriate and another photographing due to the mistake in the coil setting can be reduced.

Note that in the aforementioned embodiment, the display control unit in this specification is implemented by the display controlling function 16b of the processing circuitry 16; however, the embodiment is not limited to this structure. For example, the display control unit in this specification may be, as an alternative to the display controlling function 16b described in the embodiment, only hardware, only software, or a combination of hardware and software.

The term “processor” used in the above description means, for example, a circuit such as a central processing unit (CPU), a graphics processing unit (GPU), an application specific integrated circuit (ASIC), a programmable logic device (for example, simple programmable logic device (SPLD)), a complex programmable logic device (CPLD), or a field programmable gate array (FPGA). The processor achieves the function by reading out a computer program saved in a storage and executing the computer program. Note that instead of saving the computer program in the storage, the computer program may be directly incorporated in a circuitry of the processor. In this case, the processor achieves the function by reading out the computer program incorporated in the circuitry and executing the computer program. The processor according to the present embodiment may be configured as one circuitry or configured by combining a plurality of independent circuitries as one processor to achieve that function.

Here, the computer program to be executed by the processor is incorporated in advance in a read only memory (ROM), a storage, or the like, and provided. Note that this computer program may be recorded in a format that can be installed or executed in these devices, in a computer readable recording medium such as a CD (compact disc)-ROM, an FD (flexible disk), a CD-R (recordable), or a DVD (digital versatile disc). This computer program may be stored on a computer connected to a network such as the Internet and provided or distributed by being downloaded through the network. For example, this computer program is formed by a module including each of the aforementioned function units. As the actual hardware, the CPU reads out the computer program from the storage medium such as a ROM and executes the computer program, and thus, each module is loaded on a main storage device and generated on the main storage device.

According to at least one of the embodiments described above, the RF coil can be disposed at the proper position.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A magnetic resonance imaging system, comprising:
a magnetic resonance imaging device including a couch with a couchtop on which a subject is placed; and
an optical photographing device, wherein
the optical photographing device is configured to acquire an image including the couch, and
the magnetic resonance imaging device includes a processing circuitry configured to perform display of information expressing a position of a first RF coil disposed under or inside the couchtop on the basis of the image acquired by the optical photographing device so that a positional relation with the subject placed on the couchtop is displayed.

2. The magnetic resonance imaging system according to claim 1, wherein the processing circuitry is configured to perform display of the information expressing the position of the first RF coil in a manner of overlapping with the image of the subject acquired by the optical photographing device.

3. The magnetic resonance imaging system according to claim 1, wherein the processing circuitry is configured to perform display of the information expressing the position of the first RF coil by projecting the information onto the subject placed on the couchtop.

4. The magnetic resonance imaging system according to claim 1, wherein the processing circuitry is configured to specify the position of the first RF coil on the basis of the image acquired by the optical photographing device.

5. The magnetic resonance imaging system according to claim 4, wherein the processing circuitry is configured to specify the position of the first RF coil while the subject is not placed on the couchtop.

6. The magnetic resonance imaging system according to claim 4, wherein the processing circuitry is configured to cause the optical photographing device to acquire the image at a timing when lowering of the couchtop is detected, and specify the position of the first RF coil using the image.

7. The magnetic resonance imaging system according to claim 4, wherein the processing circuitry is configured to cause the optical photographing device to photograph continuously after the first RF coil is set, and at a timing when an operator who sets the coil goes out of a frame of an image capture region of the optical photographing device, specify the position of the first RF coil using the image obtained previously.

8. The magnetic resonance imaging system according to claim 4, wherein the processing circuitry is configured to cause the optical photographing device to photograph continuously after the first RF coil is set, and at a timing when the subject comes in a frame of the image of the couchtop, specify the position of the first RF coil using the image obtained previously.

9. The magnetic resonance imaging system according to claim 1, wherein
the first RF coil is embedded inside the couchtop, and
the processing circuitry is configured to specify the position of the first RF coil on the basis of the positional relation between the couchtop and the first RF coil.

10. The magnetic resonance imaging system according to claim 1, wherein
the first RF coil includes a plurality of elements, and
the processing circuitry is configured to perform display of information expressing a position of all or a part of the elements included in the first RF coil, within the information expressing the position of the first RF coil.

11. The magnetic resonance imaging system according to claim 1, wherein the first RF coil is an RF coil for capturing an image of a spine.

12. The magnetic resonance imaging system according to claim 1, wherein the processing circuitry is configured to further perform display of information expressing a recommended position to dispose a second RF coil that is to be disposed on the subject, so that the positional relation with the subject placed on the couchtop is displayed.

13. The magnetic resonance imaging system according to claim 12, wherein the processing circuitry is configured to determine the recommended position to dispose the second RF coil on the basis of the position of the first RF coil.

14. The magnetic resonance imaging system according to claim 12, wherein the processing circuitry is configured to perform display of the information expressing the recommended position to dispose the second RF coil in a manner of further overlapping with the image of the subject that is acquired by the optical photographing device.

15. The magnetic resonance imaging system according to claim 12, wherein
the second RF coil includes a plurality of elements, and
the processing circuitry is configured to perform display of information expressing a position of all or a part of the elements included in the second RF coil, within the information expressing the recommended position to dispose the second RF coil.

16. The magnetic resonance imaging system according to claim 12, wherein the processing circuitry is configured to perform display of the information expressing the position of the first RF coil and the information expressing the recommended position to dispose the second RF coil so that the positional relation with the second RF coil that is actually disposed on the subject is displayed.

17. A position display method executed in a magnetic resonance imaging system, the resonance imaging system including: a magnetic resonance imaging device including a couch with a couchtop on which a subject is placed; and an optical photographing device, the position display method comprising:
acquiring an image including the couchtop by the optical photographing device; and
performing, by a processing circuitry included in the magnetic resonance imaging device, display of information expressing a position of a first RF coil disposed under or inside the couchtop on the basis of the image acquired by the optical photographing device so that a positional relation with the subject placed on the couchtop is displayed.
